(19) United States
(12) Patent Application Publication
(10) Pub. No.: US 2008/0125719 A1
(43) Pub. Date: May 29, 2008

(54) Gastro-tube using a shape-retentive alloy and method of use thereof

(76) Inventor: Norikata Takuma, Kokubunji City (JP)

Correspondence Address: JORDAN AND HAMBURG LLP, 122 EAST 42ND STREET, SUITE 4000, NEW YORK, NY, 10168, US

(21) Appl. No.: 11/484,290

(22) Filed: Jul. 11, 2006

(30) Foreign Application Priority Data
Jun. 2, 2006 (JP) 2006-154568

(51) Int. Cl. A61M 25/04

(52) U.S. Cl. 604/175

(57) ABSTRACT

A gastrostomy tube of the present invention which includes a tube body formed as a cylindrical body and a leading end to which shape memory alloy pull-out prevention pieces are attached is used in combination with opening enlarging including forceps including a funnel-shaped part having a leading end divided into two, two gripping rods respectively fixed to the sides of the funnel-shaped part, and a spring part fixed to the rear end of the supporting rods.

### BACKGROUND OF THE INVENTION

The present invention relates to the provision, in the field of medicine, of fluids and nourishment to patients unable to ingest orally; more particularly, it relates to the improvement of a tube for use in gastrostomy, a type of enteral feeding.

Conventionally, patients who are suffering from severe brain damage and/or dementia, and are unable to normally ingest food orally require enteral feeding. Methods for inserting a feeding tube can be broadly classified into the following categories:

(1) passing a tube through an anatomically existing space; and
(2) creating a new route for tube insertion (e.g., gastrostomy).

Providing fluids and nutrition using percutaneous endoscopic gastrostomy (PEG) is essential for the survival of patients, and such a method depends upon a medical tool called a gastrostomy tube. When we consider the functions that are required of a gastrostomy tube, we find that they fall into three categories:

a. ease of insertion;
b. reliability as a feeding tract after insertion; and
c. ease of removal.

Among these, a. and c. are required for regular replacement of gastrostomy tubes, or reinsertion of a tube after inadvertent removal.

There is no particular medically established method for reinsertion of such a gastrostomy tube, and currently, the individual skills of the doctor in charge are relied upon. For this reason, there is the risk of mis-insertion of a tube into the abdominal cavity, causing the complications of peritonitis.

As disclosed in Laid-open Japanese Patent Application No. 2002-17866 (Tool for Maintaining Gastrostomy Hole and Guiding Gastrostomy Tube), a holding tool comprising a guide part for maintaining a gastrostomy hole and at the same time guiding a gastrostomy tube and a handle part for user operation of the guide part is known as a holding tool used when replacing such a gastromstomy tube. This holding tool is inserted, at time of removing an already inserted gastrostomy tube, alongside the gastrostomy tube.

Meanwhile, as a tube using a shape memory alloy together with medical equipment, a “medical tube” disclosed in Published Japanese Patent No. H7-106223 is a “dual-use tube comprising a tube body, a drive member made from a shape memory alloy disposed in the tube body that when heated deforms and causes the tube body to bend, and an elastic member made from a superelastic alloy that, when the drive member disposed in the tube body is not heated to at least a prescribed temperature, prevents the drive member from being plastically deformed by an external force and causing the tube to deform, and returns the tube to an initial prescribed shape,” indicating that a tube will deform driven by a shape alloy subject to temperature change.

### SUMMARY OF THE INVENTION

As described above, a PEG method is effective for feeding for patients who are unable to ingest sufficient food orally; however, a tube used in such method has several problems. More specifically, a tube needs to be regularly replaced due to change in quality caused by gastric juice. Further, if a tube is inadvertently pulled out, reinsertion at as early a stage as possible (preferably within 5 to 6 hours) is necessary. For this reason, there has been demand, as described above, for a three-stage solution to these problems with respect to a tube to be used for PEG. To repeat, these are:

a. Ease of Insertion

Experience shows that, as one might expect, a tube having as smooth a shape as possible, without any roughness, and that is thin and hard, is easily inserted;

b. Reliability as a Feeding Tract

An inner cavity allowing a viscous liquid food to pass through without difficulty is necessary, flexibility to match the movements of a patient's body is necessary, and an attachment of special shape for the end is required to prevent the tube from slipping out.

c. Ease of Removal

The quality of a gastrostomy tube will change due to gastric juice or a tube will become dirty from injected liquid food, and thus regular replacement if required. In such a case, easy removal of a tube is an essential condition.

The inventor engaged in extensive study with a view toward solving such problems, and in order to give a single tube the mutually contradictory functions described above—that is, to achieve both the function of stably preventing slipping out during use and ease of removal during replacement—invented a tube using a shape memory alloy at the leading end thereof.

Thus, a first aspect of the present invention is a gastrostomy tube using a shape memory alloy, wherein there are provided at the leading end of the cylindrical tube body, pull-out prevention pieces using a shape memory alloy selected from among a copper alloy, zinc alloy, and titanium alloy.

A second aspect of the present invention is a gastrostomy tube using a shape memory alloy according to claim 1, wherein the shape memory alloy assumes a prescribed shape at a temperature lower than normal body temperature.

A third aspect of the present invention is a gastrostomy tube using a shape memory alloy according to either claim 1 or claim 2, wherein two shape memory alloys are used so that one assumes a prescribed shape at a temperature lower than normal body temperature, and the other assumes a prescribed shape at a temperature higher than normal body temperature.

A fourth aspect of the present invention is a method for using a gastrostomy tube using a shape memory alloy, wherein an opening created between an abdominal wall and stomach wall is enlarged using opening enlarging forceps, a gastrostomy tube having, at the leading end of a cylindrical tube body, pull-out prevention pieces using a shape memory alloy selected from among a copper alloy, zinc alloy, and titanium alloy is inserted into the forceps, the forceps are then pulled out and the shape memory alloy pull-out prevention pieces unfold in the stomach due to the body temperature, thereby preventing pull-out of the tube.

A fifth aspect of the present invention is a method for using a gastrostomy tube using a shape memory alloy according to claim 4, wherein the opening enlarging forceps comprise a funnel-shaped part having a leading end divided into two, two gripping rods respectively fixed to the sides of the funnel-shaped part, and a spring part fixed to the rear ends of the gripping rods, the two gripping rods being fixed to each other roughly at the center thereof via a hinge so as to freely open and close in the lateral direction.

Because the tools for reinserting a gastrostomy tube of the present invention basically are a gastrostomy tube used in combination with opening enlarging forceps, there is no difference in operation depending on operator, enabling uniform operation.

With respect to operating method, for regular replacement of gastrostomy tubes, opening enlarging forceps are used, so that the leading end thereof spans the entire length of the opening along the old, already inserted gastrostomy tube, and then the funnel parts of the forceps are enlarged; thereafter, pull-out prevention pieces at the leading end of the gastrostomy tube are cooled, enabling easy pull-out; therefore, the pull-out operation is easy.

With respect to a reinsertion operation when a gastrostomy tube is inadvertently pulled out, a sounding rod is used and a tube is inserted into the opening between an abdominal wall and stomach wall, and thereafter opening enlarging forceps are used, with the effect that an operation identical to the above can be easily performed.

### BRIEF DESCRIPTION OF THE DRAWINGS

FIG. 1 is a diagram for explaining a gastrostomy tube of the present invention;

FIG. 2a is a diagram for explaining a state where the pull-out prevention pieces at the leading end of FIG. 1 are expanded;

FIG. 2b is a diagram for explaining a state where the pull-out prevention pieces at the leading end of FIG. 1 are expanded;

FIG. 3 is an oblique view showing opening enlarging forceps as a gastrostomy tube reinsertion tool of the present invention;

FIG. 4a is an oblique view of the funnel parts of FIG. 3;

FIG. 4b is a cross-sectional view thereof;

FIG. 5a is a diagram for explaining means for using opening enlarging forceps as a gastrostomy tube reinsertion tool;

FIG. 5b is a diagram for explaining means for using opening enlarging forceps as a gastrostomy tube reinsertion tool;

FIG. 5c is a diagram for explaining means for using opening enlarging forceps as a gastrostomy tube reinsertion tool;

FIG. 5 d is a diagram for explaining means for using opening enlarging forceps as a gastrostomy tube reinsertion tool;

FIG. 5 e is a diagram for explaining means for using opening enlarging forceps as a gastrostomy tube reinsertion tool;

FIG. 5 f is a diagram for explaining means for using opening enlarging forceps as a gastrostomy tube reinsertion tool;

FIG. 6a is a diagram for explaining means for using opening enlarging forceps as a gastrostomy tube reinsertion tool;

FIG. 6b is a diagram for explaining means for using opening enlarging forceps as a gastrostomy tube reinsertion tool; and

FIG. 6c is a diagram for explaining means for using opening enlarging forceps as a gastrostomy tube reinsertion tool.

Alternatively, an ultrasonic oscillator can be attached near the gastrostomy tube leading end, enabling a user to find an opening in a stomach wall while viewing the image thereof.

### DETAILED DESCRIPTION OF THE INVENTION

There are two types of gastrostomy tubes according to the present invention, depending on how shape memory alloys are used. Type A uses one shape memory alloy for pull-out prevention pieces, which assume a prescribed shape at a temperature lower than normal body temperature, whereas type B uses two memory alloys for pull-out prevention pieces, of which one assumes a prescribed shape at a temperature lower than normal body temperature, and the other assumes a prescribed shape at a temperature higher than normal body temperature.

First, an explanation will be given for type A. The shape memory alloy used for this type is set so as to open in a petal-shape at a temperature of, for example, 25° C., which is lower than body temperature. When the type A is used, the shape memory alloy of pull-out prevention pieces attached to the leading end of a gastrostomy tube are narrowed in cold water to be sufficiently small so to pass through the opening enlarging forceps.

The opening enlarging forceps used in the present invention are used as a tool for reinserting a gastrostomy tube in combination with a sounding rod previously presented as a Gastrostomy Tube Reinsertion Tool of Laid-open Japanese Patent Application No. 2005-70907. For example, the diameter of the forceps lower end can accommodate a sounding rod with a diameter of 3 mm but does not accommodate a gastrostomy tube thicker than the sounding rod, requiring operation of the forceps to slightly enlarge the opening for insertion of the tube.

After an opening is enlarged and a gastrostomy tube is inserted into a stomach cavity, body temperature is transmitted to the pull-out prevention pieces at the tube leading end; when the temperature rises to 25° C., the shape memory alloy that had been made narrow expands to a prescribed petal-shape, functioning to prevent tube pull-out.

To remove such a type A gastrostomy tube, because when warmed by body temperature, the expanded pull-out prevention pieces at the leading end prevent pull-out of the tube, this section first needs to be deformed to be sufficiently thin to pass through the opening, and thus the following means is used.

First, when the opening enlarging forceps used to insert a tube is attached to the tube again, and the forceps are inserted along the tube in the opening, the forceps stop when they come in contact with the expanded pull-out prevention pieces at the leading end, and cooling water prepared in advance is dripped inside of the tube or along the outside of the tube, thereby cooling the pull-out prevention pieces at the tube leading end.

By infusion of the cooling water, when the temperature of the pull-out prevention pieces at the end drops to 25° C. or less, which is lower than body temperature, the forceps are operated so that tube is slightly pulled into the forceps, and the forceps fold up the pull-out prevention pieces opened at the lower end of the forceps. That portion of the pull-out prevention pieces, which have been cooled to 25° C. or lower, sandwiched by the forceps is narrowed to the extent sandwiched by the forceps, and will not expand, and such operation is repeated and the tube removed.

The type B tube, which is another gastrostomy tube of the present invention, uses two memory alloys for pull-out prevention pieces, of which one assumes a prescribed shape at a temperate lower than normal body temperature (for example 25° C.), and the other assumes a prescribed shape at a temperature higher than normal body temperature (for example 43° C.).

To insert this type B tube into an opening, the pull-out prevention pieces at the tube leading end are made narrower in cooling water, as with the type A, and inserted into a stomach via an opening secured by the opening enlarging forceps. When body temperature is transmitted to the pull-out prevention pieces in such a state and raises the temperature thereof to 25° C., the pull-out prevention pieces open due to the effects from the shape memory alloy for a lower temperature, preventing pull-out of the tube.

For regular replacement of gastrostomy tubes, a tube can be removed as follows. As with a type A tube, the opening enlarging forceps are inserted along the tube, and the forceps lower end proceeds until coming in contact with the expanded pull-out prevention pieces.

Next, when the forceps lower end comes in contact with the pull-out prevention pieces, warm water of 43° C. to 44° C. prepared in advance is dripped along the outer periphery of the tube, and the pull-out prevention pieces are warmed to 43° C. As a result, due to the operations of the shape memory alloy for a higher temperature, the expanded pull-out prevention pieces assume a closed shape, thereby facilitating pull-out of the tube.

In this case, the temperature range of 43° C. to 44° C. represents a temperature range that body temperature will never reach even in the case of a fever as well as a temperature that the human body can stand. In the present invention, the lower temperature is set at 25° C. and the higher temperature at 43° C. It goes without saying that such temperature range can be adjusted to facilitate operations.

With such a constitution, by selecting measures matching the properties of the shape memory alloys used for the present invention, the mutually contradictory demands for ease of insertion of a gastrostomy tube, prevention of pull-out after insertion, and ease of removal when removal is required can be addressed.

With respect to a method for using a gastrostomy tube of the present invention, a gastrostomy tube to the leading end of which is attached a shape memory alloy that assumes a prescribed shape at a temperature lower than normal body temperature is used, an opening created between an abdominal wall and stomach wall is enlarged by opening enlarging forceps and a gastrostomy tube at the leading end of which the shape memory alloy is provided is inserted into a stomach, and when warmed by body temperature so as to reach a prescribed temperature (for example, 25° C.), the shape memory alloy pull-out prevention pieces unfold into a prescribed shape such as a petal-shape, thereby preventing pull-out of the tube.

In another method for use of the present invention, two shape memory alloys are used in combination. One of them unfolds to assume a prescribed petal-shape at a temperature lower than normal body temperature (for example, 25° C.), and the other has a property causing the unfolded pull-out prevention pieces to shut and shrink at a temperature higher than normal body temperature (for example, 43° C.).

With a combination of such two shape memory alloys, the alloys are made narrow using cooling water and when in the stomach their temperature reaches 25° C., the pull-out prevention pieces expand due to the operations of the alloy for a lower temperature, and if warm water of 43° C. is applied, the opened pull-out prevention pieces close.

A detailed explanation will be given for the present invention with reference to the drawings. However, the scope of the present invention should not be construed as being limited thereto.

### Embodiment 1

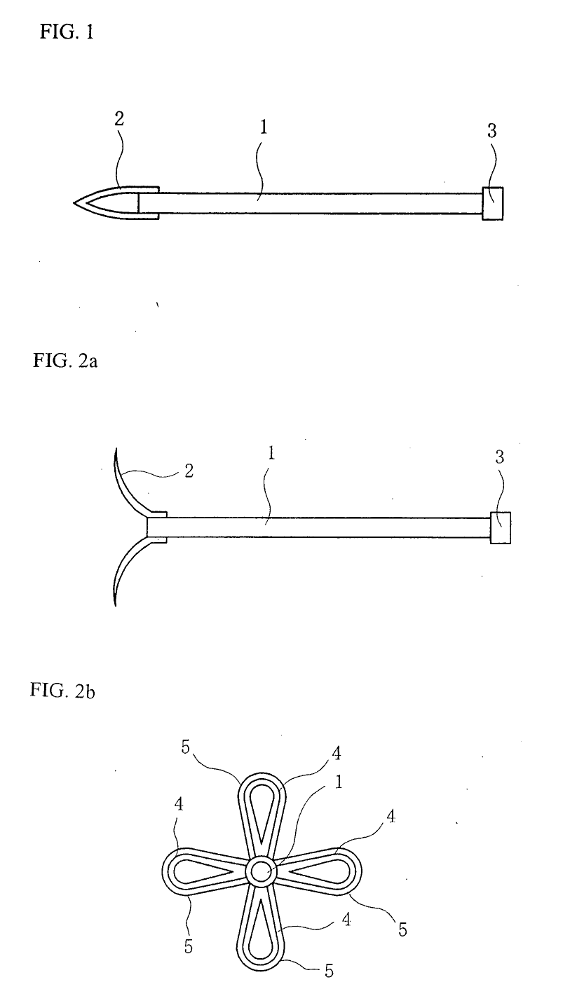

FIG. 1 is an oblique view showing a gastrostomy tube 1 of the present invention. The gastrostomy tube 1 is formed in a cylindrical body having an outer diameter of about 7 mm to 9 mm and a smooth circular cross-sectional outline, and when combined with a lower end of a funnel-shaped part 9 of opening enlarging forceps 8, the tube is in close contact therewith and can slide smoothly in the vertical direction.

The gastrostomy tube 1 has a leading end on which a Cu—Zn shape memory alloy wire 4 having a diameter of 2 mm is folded in a roughly oval shape, and around the wire, four pull-out prevention pieces 2 coated with a transparent resin material 5 are attached integrally therewith. Further, the gastrostomy tube 1 has a rear end on which a joint 3 is provided so that a syringe (not shown in the drawings) can be connected thereto.

FIGS. 2(a) to 2(b) are diagrams for explaining a state where the four pull-out prevention pieces 2 are warmed by body temperature within a stomach, and are fixed in a state of an outwardly expanded petal-shape. In the present embodiment, the pull-out prevention pieces 2 are set in advance so as to expand at a temperature from 20° C. to 30° C., and more preferably at a temperature around 25° C.

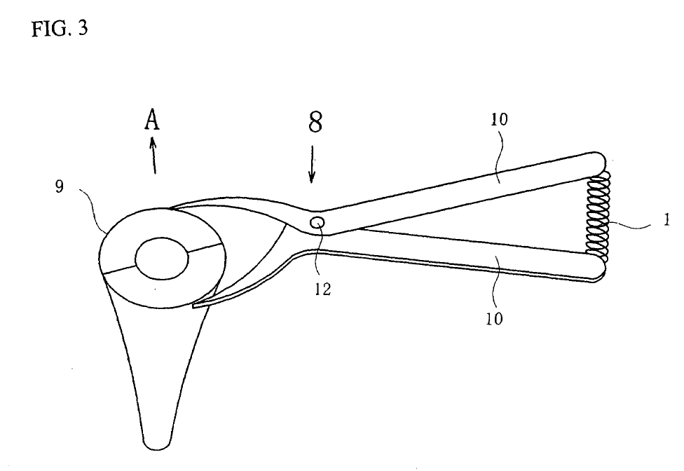

FIG. 3 is an oblique view showing the opening enlarging forceps 8. The opening enlarging forceps 8 comprise a funnel-shaped part 9 having a leading end divided into two, two gripping rods 10 respectively fixed to the sides of the funnel-shaped part 9, and a spring part 11 fixed to the rear ends of the gripping rods, the two gripping rods being fixed to each other roughly at the center thereof via a hinge 12 so as to freely open and close.

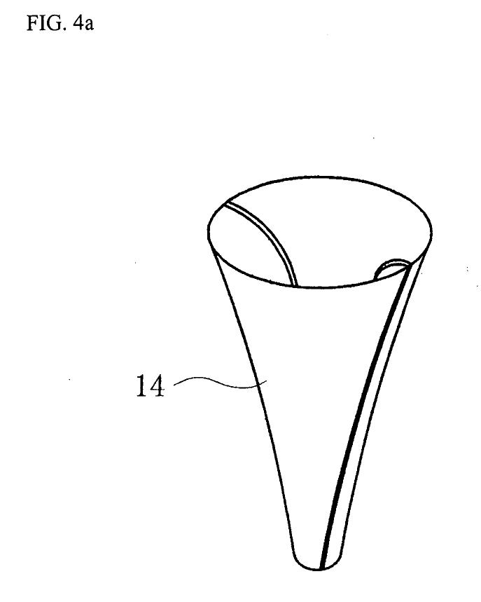

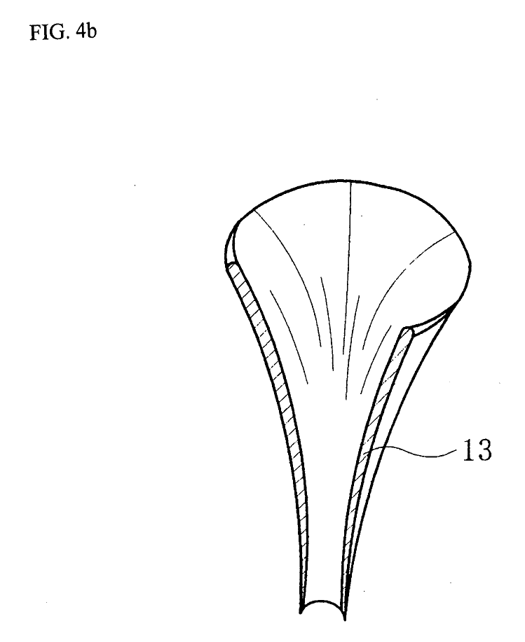

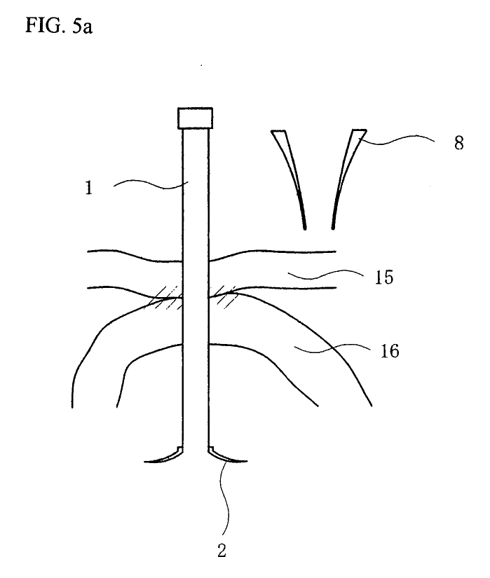

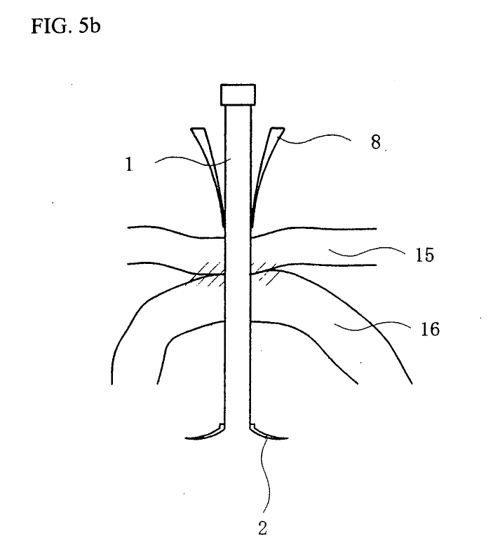

The funnel-shaped part 9 is divided into right and left funnel bodies as shown in FIG. 4, and forms a long thin shape that tapers in the leading end direction, designed so that the inner diameter of the leading end is slightly larger than the outer diameter of a sounding rod for reinsertion (not shown in the drawings).

When a user grips the two gripping rods 10, the spring part 11 attached to the rear ends of the gripping rods contracts, causing the right and left funnel bodies 13, 14 fixed at the leading ends of the gripping rods 10 to open. As a result, the opening enlarging forceps 8 can be attached so as to sandwich the sounding rod for reinsertion from outside.

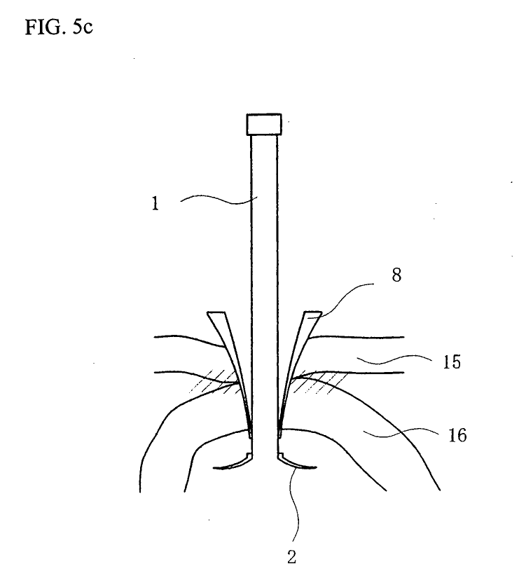

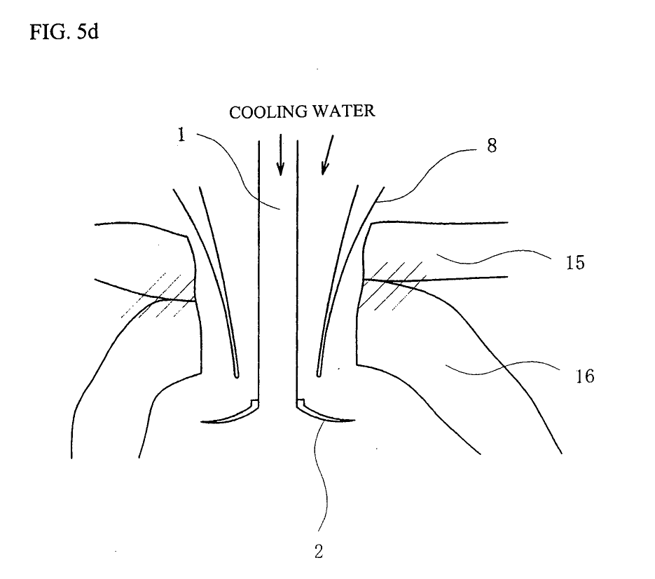

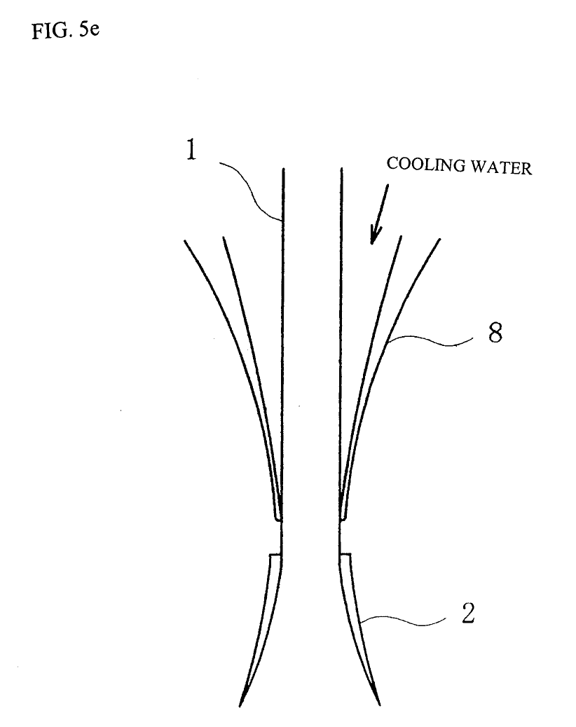

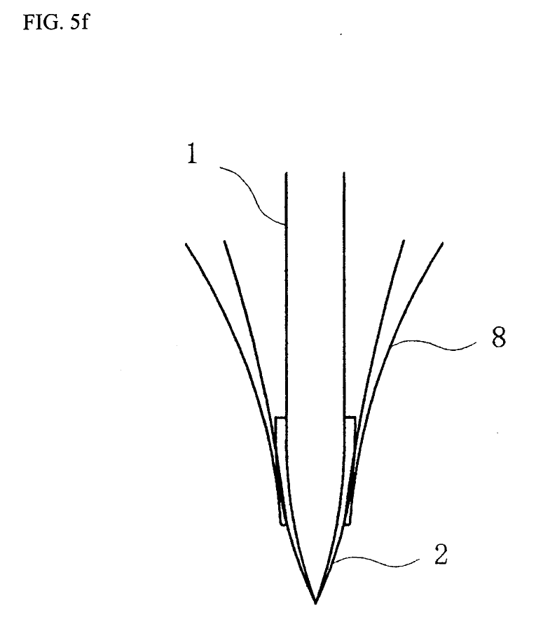

The method for using the opening enlarging forceps 8 to replace a gastrostomy tube 1 on a regular basis as shown in FIGS. 5a to 5c differs somewhat from the method used for reinsertion of a tube after inadvertent pull-out.

In the case of regular replacement, the pull-out prevention pieces 2 attached at the leading end of the inserted gastrostomy tube 1 are designed so that, when the shape memory alloy wire 3 coated with a resin is warmed by body temperature, they start expanding when the temperature exceeds 23° C., expand 180 degrees at around 25° C., and then stop expanding, thereby preventing pull-out of the gastrostomy tube 1 once the wire expands inside the stomach.

When the tube 1 thus configured is to be removed, as shown in FIG. 5, the opening enlarging forceps 8 are moved near the currently inserted gastrostomy tube 1. Then, with the tube 1 serving as guide as shown in FIG. 5b, the opening enlarging forceps are downwardly slid along the tube.

In this case, because the gastrostomy tube 1 has an outer diameter of only 7 mm to 9 mm, the lower end of the funnel part 9 of the opening enlarging forceps 8, which is formed so as to be in close contact with a sounding rod having an outer diameter of 3 mm to 4 mm, will naturally be slightly open. In this state, the funnel part 9 is carefully pressed down along the gastrostomy tube 1 so that the funnel part 9 is inserted so as to span the entire length of the opening as shown in FIG. 5c.

Next, as shown in FIG. 5d, the funnel part 9 of the opening enlarging forceps 8 and the unfolded pull-out prevention pieces of the gastrostomy tube 1 are cooled. From the point, the process differs between type A and type B.

In the case of type A (the pull-out prevention pieces unfold at a temperature of 25° C.), if cooled to a temperature of 25° C. or lower, the pull-out prevention pieces will not return to the set shape even when deformed by an external force. More specifically, as shown in FIGS. 5c and 5f, by repeatedly pulling a section of the pull-out prevention pieces into the lower end of the funnel part 9 of the opening enlarging forceps and closing upon and closing such section with the forceps, in the end the pull-out prevention pieces are pulled within the funnel and the tube is removed.

In the case of type B (two shape memory alloys are used in combination), in a stomach cavity maintained at body temperature, the pull-out prevention pieces unfold due to the operations of a shape memory alloy for a lower temperature (25° C.). In addition, due to the operations of a shape memory alloy for a higher temperature set so that when warmed by water at 43° C., the pull-out prevention pieces shut and shrink, the unfolded pull-out prevention pieces assume a shutting shape, facilitating removal via the funnel.

To determine whether insertion into the stomach has been made, a syringe 20 is attached to the joint 3 at the rear end of the gastrostomy tube 1, and when gastric juice 19 in the stomach is sucked from an opening 4 near the leading end of the gastrostomy tube body 2, confirmation is made. Inflow of the gastric juice into the syringe indicates that the leading end of the gastrostomy tube 1 has reached the inside of the stomach.

For a case of reinsertion of a gastrostomy tube after inadvertent pulling out, the tube is reinserted by using a sounding rod, (not shown in the drawings) used in combination with the opening enlarging forceps 8, that has an outer diameter of 3 mm to 4 mm, to secure the route (which is usually somewhat curved or shrunken) from an opening 18 in an abdominal wall to the stomach cavity.

Next, when the sounding rod has properly passed through the route of the opening 18, with the sounding rod as guide, the lower end of the funnel part 9 of the opening enlarging forceps 8 is inserted into the stomach cavity.

In this case, because the lower end inner diameter of the funnel part 9 of the forceps 8 is formed so as to allow the funnel part 9 to be in close contact with and slide on the outer diameter of the sounding rod, thereafter, by carefully inserting the funnel along the sounding rod, the opening 18 can be secured along its entire route using the funnel part 9. By pulling out the sounding rod and expanding the funnel part 9, the entire length of the opening 18 is sufficiently expanded to allow the safe and reliable insertion of a new gastrostomy tube 1.

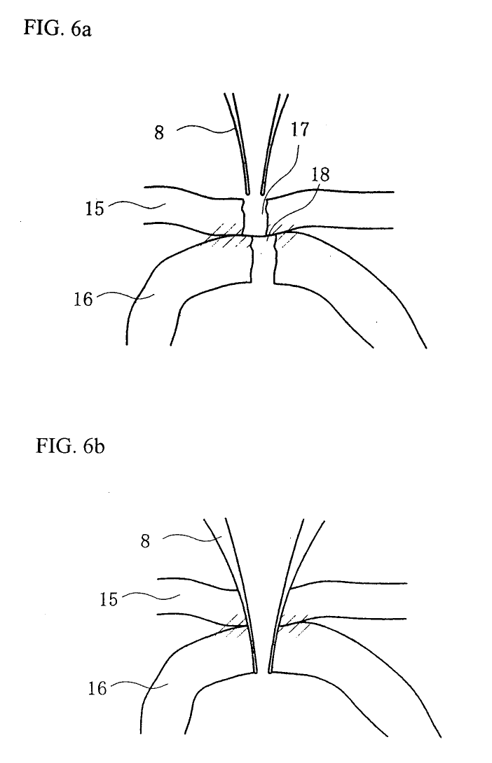

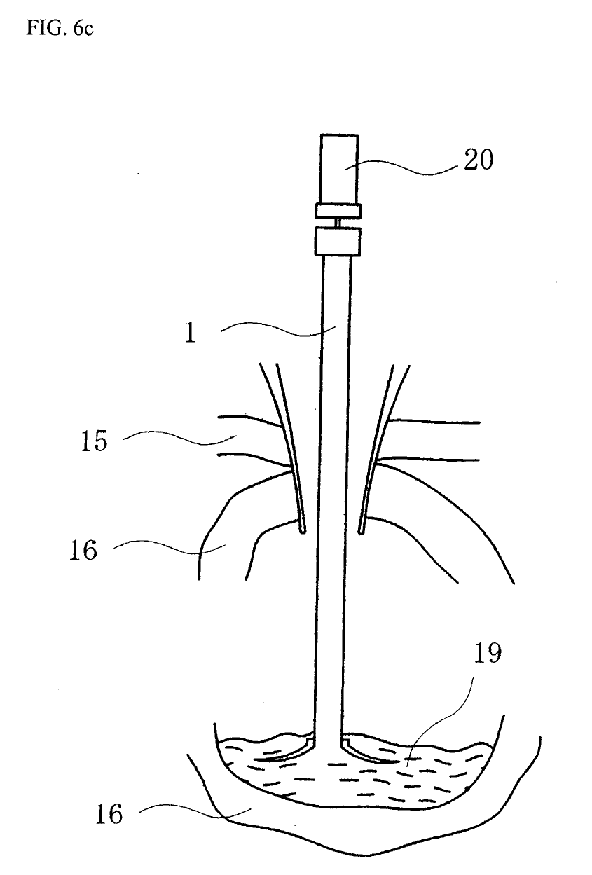

On the other hand, when the gastrostomy tube 1 is to be pulled out from the stomach, the opening enlarging forceps 8 is attached to the gastrostomy tube 1 by means as shown in FIG. 5a, 5b. First, the opening enlarging forceps 8 are moved near a gastrostomy tube inserted into a stomach (FIG. 5a), and then the right and left funnel parts 13, 14 of the opening enlarging forceps 8 are opened so as to sandwich the gastrostomy tube body 2 from the outside (FIG. 6b).

The leading ends of the right and left funnel parts 13, 14 are configured so as to be in close contact with and vertically slide on the outer surface of the gastrostomy tube 1. For this reason, when the funnel parts 13, 14 are lowered in order (FIG. 6a, 6b), the leading ends thereof pass through an abdominal wall 15, adhesion part 18, and opening 17 of the stomach wall 16 to be inserted into the stomach (FIG. 6c).

After confirming that the leading ends of the funnel parts 13, 14 have been inserted into the stomach, a user grips the gripping rods of the opening enlarging forceps 8, opens the right and left funnel parts 13, 14, and while maintaining this opened state, causes cold water to flow little by little from above along the exterior or within the interior of the gastrostomy tube 1, thereby cooling the pull-out prevention pieces 2 at the leading end of the gastrostomy tube 1.

The pull-out prevention pieces 2 are formed from a shape memory alloy, and the present invention is configured such that the pull-out prevention pieces are set so as to unfold at a temperature of 25° C. or higher, preventing pull-out of a tube at body temperature.

Thus, the pull-out prevention pieces 2 are cooled to 25° C. or lower, and the pull-out prevention pieces 2, which had assumed an unfolded shape, are shrunk to a size sufficiently small to allow passage by some method through the lower end of the funnel part of the opening enlarging forceps. In such a manner, a tube is removed while the temperature of the pull-out prevention pieces 2 is 25° C. or lower.

More specifically, the funnel part of the opening enlarging forceps 8 is carefully inserted into the opening along the gastrostomy tube, and lowered until the funnel part lower end comes in contact with the unfolded pull-out prevention pieces 2. When the pull-out prevention pieces 2 are reached, insertion is temporarily stopped, and prepared cold water is dripped inside and outside of the tube so as to cool the unfolded pull-out prevention pieces to 25° C. or lower. At this time, if the forceps are operated to slightly expand the opening, a gap is created between the tube and funnel parts, and this is desirable for cooling the shape memory alloy of the pull-out prevention pieces at the lower end using the dripped cold water.

In this case, if unfolded pull-out prevention pieces 2 are cooled to 25° C. or lower, even if pulled into the funnel part lower end bit by bit and crushed, the pull-out prevention pieces will not unfold again, allowing in the end removal of the tube.

### Embodiment 2

Another gastrostomy tube of the present invention is configured such that two shape memory alloys are used in combination for the pull-out prevention pieces; one assumes a petal shape at a temperature lower than normal body temperature (for example, 25° C.), and the other assumes a closed-petal shape at a temperature higher than normal body temperature (for example, 43° C.).

To insert such a gastrostomy tube into an opening, as in embodiment 1, the pull-out prevention pieces at the tube leading end are narrowed in cold water to enable passage through the opening secured by the opening enlarging forceps. If in this state the body temperature is transmitted to the pull-out prevention pieces so that the temperature thereof reaches 25° C., the pull-out prevention pieces open due to the operations of the shape memory alloy for a lower temperature, thereby preventing pull-out of the tube.

At time of regular replacement and the like of gastrostomy tubes, as in embodiment 1, a tube is removed by inserting the opening enlarging forceps 8 along the tube, and making the forceps lower end proceed until coming in contact with the expanded pull-out prevention pieces 2.

Next, when the forceps lower end comes in contact with the pull-out prevention pieces 2, prepared warm water of 43° C. to 44° C. is dripped along the outer periphery of the tube, and when the pull-out prevention pieces 2 are warmed to 43° C., due to the operations of the shape memory alloy for a higher temperature, the expanded pull-out prevention pieces assume a closed shape, thereby facilitating pull-out of the tube.

In this case, 43° C. to 44° C. represents a temperature range that body temperature will not reach, even in cases of a fever. Further, it is a temperature that the human body can withstand. In the present invention, the lower temperature is set at 25° C. and the higher temperature at 43° C. It goes without saying that the temperature range can be adjusted so as to facilitate operation.

### LEGENDS

1 . . . Gastrostomy tube
2 . . . Pull-out prevention pieces
3 . . . Joint
4 . . . Wire
5 . . . Resin material
8 . . . Opening enlarging forceps
9 . . . Funnel-shaped part
10 . . . Gripping rods
11 . . . Spring part
12 . . . Hinge
13 . . . Right funnel body
14 . . . Left funnel body
15 . . . Abdominal wall
16 . . . Stomach wall
17 . . . Opening
18 . . . Adhesion part of abdominal wall and stomach wall
19 . . . Gastric juice
20 . . . Syringe

1.-5. (canceled)

6. A gastrostomy tube comprising a cylindrical tube having at a leading end thereof components for preventing inadvertent pulling out of the gastrostomy tube from the stomach of a patient, the components being comprised of a copper-, zinc- and/or titanium-containing shape memory alloy.

7. A gastrostomy tube in accordance with claim 7, wherein the components assume a closed configuration when at predetermined first temperatures below normal body temperature and an open configuration when at predetermined second temperatures below normal body temperature, the second temperatures being higher than the first temperatures.

8. A gastrostomy tube in accordance with claim 7, wherein the components are comprised of two of the shape memory alloys and assume a closed position when at predetermined first temperatures below normal body temperature, assume an open position at predetermined second temperatures below normal body temperature, the second temperatures being higher than the first temperatures, and assume the closed position when at third temperatures, the third temperatures being higher than body fever temperature.

9. A gastrostomy tube in accordance with claim 7, wherein the first temperatures are no higher than 25° C. and the second temperatures are above 25° C.

10. A gastrostomy tube in accordance with claim 8, wherein the first temperatures are below 25° C., the second temperatures are in a range above 25° C. to below 43° C. and the third temperatures are 43° C. and above.

11. A gastrostomy tube in accordance with claim 10, wherein the third temperatures are 43° C. to 44° C.

12. A method of using a gastrostomy tube of claim 6 for a patient having an opening passing through abdominal wall and stomach wall of
